(19) United States
(12) Patent Application Publication
(10) Pub. No.: US 2016/0375060 A1
(43) Pub. Date: Dec. 29, 2016

(54) Methods of Treating Glioblastoma Multiforme by T Cell Therapy

(71) Applicant: Memorial Sloan Kettering Cancer Center, New York, NY (US)

(72) Inventors: Richard John O'Reilly, Roxbury, CT (US); Aisha Nasreen Hasan, New York, NY (US); Guenther Koehne, New York, NY (US); Ekaterina Doubrovina, Bronx, NY (US)

(73) Assignee: Memorial Sloan Kettering Cancer Center, New York, NY (US)

(21) Appl. No.: 15/191,724

(22) Filed: Jun. 24, 2016

Related U.S. Application Data
(60) Provisional application No. 62/185,563, filed on Jun. 27, 2015.

(51) Int. Cl. A61K 35/17; C12N 5/0783; A61K 9/00

(52) U.S. Cl. CPC A61K 35/17; A61K 9/0019; C12N 5/0636; A61K 2035/124

(57) ABSTRACT

Disclosed herein are methods of treating glioblastoma multiforme (GBM) in a human patient in need thereof, comprising administering to the human patient a population of allogeneic T cells comprising CMV (cytomegalovirus)-specific T cells.

### CROSS-REFERENCE TO RELATED APPLICATIONS

This application claims the benefit of U.S. Provisional Patent Application No. 62/185,563, filed Jun. 27, 2015, which is incorporated by reference herein in its entirety.

### FIELD

Disclosed herein are methods of treating glioblastoma multiforme (GBM) in a human patient in need thereof, comprising administering to the human patient a population of allogeneic T cells comprising CMV (cytomegalovirus)-specific T cells.

### BACKGROUND

Glioblastoma multiforme (GBM) is the most aggressive of the gliomas, a collection of tumors arising from glia or their precursors. In the recent few years, multiple investigators have confirmed the presence of cytomegalovirus (CMV) in GBM, and multiple CMV gene products have been implicated in biologically relevant GBM signaling pathways. Approximately 95% of GBM tumors express CMV antigen(s).

Most patients who are diagnosed with GBM have a mean survival less than two years. GBM is very difficult to treat due to several complicating factors (Lawson et al., J Neurooncol 83:61-70): (1) the tumor cells are very resistant to conventional therapies; (2) the brain is susceptible to damage due to conventional therapy; (3) the brain has a very limited capacity to repair itself; and (4) many drugs cannot cross the blood-brain barrier to act on the tumor.

Therefore there is a need for methods of treating GBM in human patients.

Citation of a reference herein shall not be construed as an admission that such is prior art to the present disclosure.

### SUMMARY OF THE INVENTION

The present invention relates to methods of treating glioblastoma multiforme (GBM) in a human patient using T cell therapy.

In one aspect, provided herein are methods of treating GBM in a human patient in need thereof, comprising administering to the human patient a population of allogeneic T cells comprising CMV-specific T cells.

In various embodiments, the population of allogeneic T cells that is administered to the human patient is restricted by an HLA allele shared with cells of the GBM.

In certain embodiments, preferably in addition to being restricted by an HLA allele shared with cells of the GBM, the population of allogeneic T cells comprising CMV-specific T cells shares at least 2 out of 8 HLA alleles (e.g., two HLA-A alleles, two HLA-B alleles, two HLA-C alleles, and two HLA-DR alleles) with cells of the GBM.

In specific embodiments, the methods of treating GBM described herein further comprise prior to the administering step a step of ascertaining at least one HLA allele of cells of the GBM by high-resolution typing.

In various embodiments, the methods of treating GBM further comprise prior to the administering step a step of generating the population of allogeneic T cells in vitro.

In certain embodiments, the step of generating the population of allogeneic T cells in vitro comprises sensitizing (i.e., stimulating) allogeneic T cells to one or more CMV antigens so as to produce CMV-specific T cells.

In certain embodiments, the step of generating the population of allogeneic T cells in vitro comprises sensitizing allogeneic T cells using dendritic cells (preferably, the dendritic cells are derived from the donor of allogeneic T cells). In specific embodiments, the step of sensitizing allogeneic T cells using dendritic cells comprises loading the dendritic cells with at least one immunogenic peptide derived from one or more CMV antigens. In specific embodiments, the step of sensitizing allogeneic T cells using dendritic cells comprises loading the dendritic cells with a pool of overlapping peptides derived from one or more CMV antigens.

In certain embodiments, the step of generating the population of allogeneic T cells in vitro comprises sensitizing allogeneic T cells using cytokine-activated monocytes (preferably, the cytokine-activated monocytes are derived from the donor of allogeneic T cells). In specific embodiments, the step of sensitizing allogeneic T cells using cytokine-activated monocytes comprises loading the cytokine-activated monocytes with at least one immunogenic peptide derived from one or more CMV antigens. In specific embodiments, the step of sensitizing allogeneic T cells using cytokine-activated monocytes comprises loading the cytokine-activated monocytes with a pool of overlapping peptides derived from one or more CMV antigens.

In certain embodiments, the step of generating the population of allogeneic T cells in vitro comprises sensitizing allogeneic T cells using peripheral blood mononuclear cells (preferably, the peripheral blood mononuclear cells are derived from the donor of allogeneic T cells). In specific embodiments, the step of sensitizing allogeneic T cells using peripheral blood mononuclear cells comprises loading the peripheral blood mononuclear cells with at least one immunogenic peptide derived from one or more CMV antigens. In specific embodiments, the step of sensitizing allogeneic T cells using peripheral blood mononuclear cells comprises loading the peripheral blood mononuclear cells with a pool of overlapping peptides derived from one or more CMV antigens.

In certain embodiments, the step of generating the population of allogeneic T cells in vitro comprises sensitizing allogeneic T cells using an EBV-transformed B lymphocyte cell line (EBV-BLCL). In specific embodiments, the step of sensitizing allogeneic T cells using an EBV-BLCL comprises loading the EBV-BLCL cells with at least one immunogenic peptide derived from one or more CMV antigens. In specific embodiments, the step of sensitizing allogeneic T cells using an EBV-BLCL comprises loading the EBV-BLCL cells with a pool of overlapping peptides derived from one or more CMV antigens.

In certain embodiments, the step of generating the population of allogeneic T cells in vitro comprises sensitizing allogeneic T cells using artificial antigen-presenting cells (AAPCs). In specific embodiments, the step of sensitizing allogeneic T cells using AAPCs comprises loading the AAPCs with at least one immunogenic peptide derived from one or more CMV antigens. In specific embodiments, the step of sensitizing allogeneic T cells using AAPCs comprises loading the AAPCs with a pool of overlapping peptides derived from one or more CMV antigens. In specific embodiments, the step of sensitizing allogeneic T cells using AAPCs comprises engineering the AAPCs to express at least one immunogenic CMV peptide or protein in the AAPCs.

In a specific embodiment, the pool of overlapping peptides is a pool of overlapping pentadecapeptides.

In specific embodiments, the methods of treating GBM described herein further comprise, after sensitizing, cryopreserving the allogeneic T cells.

In specific embodiments, the methods of treating GBM described herein further comprise, before the administering step, steps of thawing cryopreserved CMV-antigen sensitized allogeneic T cells, and expanding the allogeneic T cells in vitro, to produce the population of allogeneic T cells.

In certain embodiments, the methods of treating GBM described herein further comprise, before the administering step, a step of thawing a cryopreserved form of the population of allogeneic T cells.

In various embodiments, the population of allogeneic T cells is derived from a T cell line. In certain embodiments, the methods of treating GBM described herein further comprise, before the administering step, a step of selecting the T cell line from a bank of a plurality of cryopreserved T cell lines (preferably each comprising CMV-specific T cells). In certain embodiments, the methods of treating GBM described herein further comprise, before the administering step, a step of thawing a cryopreserved form of the T cell line. In specific embodiments, the methods of treating GBM described herein further comprises, before the administering step, a step of expanding the T cell line (for example, after thawing a cryopreserved form of the T cell line) in vitro.

In specific embodiments, the CMV-specific T cells administered in accordance with the methods described herein recognize CMVpp65.

In specific embodiments, the CMV-specific T cells administered in accordance with the methods described herein recognize CMV IE1.

In certain embodiments, the administering is by infusion of the population of allogeneic T cells. In some embodiments, the infusion is bolus intravenous infusion.

In certain embodiments, the administering comprises administering at least about 1×10⁵ T cells of the population of allogeneic T cells per kg per dose per week to the human patient. In some embodiments, the administering comprises administering about 1×10⁶ to about 2×10⁶ T cells of the population of allogeneic T cells per kg per dose per week to the human patient. In a specific embodiment, the administering comprises administering about 1×10⁶ T cells of the population of allogeneic T cells per kg per dose per week to the human patient. In another specific embodiment, the administering comprises administering about 2×10⁶ T cells of the population of allogeneic T cells per kg per dose per week to the human patient.

In certain embodiments, the methods of treating GBM described herein comprise administering at least 2 doses of the population of allogeneic T cells to the human patient. In specific embodiments, the methods of treating GBM described herein comprise administering 2, 3, 4, 5, or 6 doses of the population of allogeneic T cells to the human patient.

In certain embodiments, the methods of treating GBM described herein comprise administering a first cycle of one dose per week of the population of allogeneic T cells for 3 consecutive weeks followed by a washout period during which no dose of the population of allogeneic T cells is administered, followed by a second cycle of the one dose per week of the population of allogeneic T cells for 3 consecutive weeks. In specific embodiments, the methods of treating GBM described herein comprise administering two, three, four, five, or six cycles of one dose per week of the population of allogeneic T cells for 3 consecutive weeks, each cycle separated by a washout period during which no dose of the population of allogeneic T cells is administered. In a specific embodiment, the washout period is about three weeks.

In certain embodiments, the methods of treating GBM further comprise, after administering to the human patient the population of allogeneic T cells, administering to the human patient a second population of allogeneic T cells comprising CMV-specific T cells; wherein the second population of allogeneic T cells is restricted by a different HLA allele shared with cells of the GBM. In a specific embodiment, the methods of treating GBM comprise administering a first cycle of one dose per week of the population of allogeneic T cells for 3 consecutive weeks followed by a washout period during which no dose of the population of allogeneic T cells is administered, followed by a second cycle of one dose per week of the second population of allogeneic T cells for 3 consecutive weeks. In a further specific embodiment, the washout period is about three weeks. In certain embodiments, the human patient has no response, an incomplete response, or a suboptimal response (i.e., the human patient may still have a substantial benefit from continuing treatment, but has reduced chances of optimal long-term outcomes) after administering the population of allogeneic T cells and prior to administering the second population of allogeneic T cells.

### DETAILED DESCRIPTION

The present invention relates to methods of treating glioblastoma multiforme (GBM) in a human patient. The invention provides a T cell therapy method that is effective in treating GBM in a human patient with low or no toxicity.

In one aspect, provided herein are methods of treating GBM in a human patient in need thereof, comprising administering to the human patient a population of allogeneic T cells comprising CMV (cytomegalovirus)-specific T cells.

### A Population of Allogeneic T Cells Restricted by an Shared HLA Allele With Cells of the GBM

According to the invention, a population of allogeneic T cells comprising CMV-specific T cells is administered to the human patient. Preferably, the population of allogeneic T cells has demonstrated anti-CMV cytotoxic activity, measured by a method known in the art (for example, as described in Trivedi et al., 2005, Blood 105:2793-2801; or Hasan et al., 2009, J Immunol 183: 2837-2850).

In a specific embodiment, the population of allogeneic T cells that is administered to the human patient is restricted by an HLA allele shared with cells of the GBM. In some embodiments, this HLA allele restriction is ensured by ascertaining the HLA assignment of cells of the GBM, and selecting a population of allogeneic T cells comprising CMV-specific T cells (or a T cell line from which to derive the population of allogeneic T cells) restricted by an HLA allele of such cells. In other embodiments, when ascertaining the HLA assignment of cells of the GBM is not possible (or is possible but not performed), this HLA allele restriction is ensured by ascertaining the HLA assignment of the human patient (e.g., by using non-tumorous cells or tissue from the human patient), and selecting a population of allogeneic T cells comprising CMV-specific T cells (or a T cell line from which to derive the population of allogeneic T cells) restricted by an HLA allele of the human patient.

In some embodiments of ascertaining an HLA assignment, at least 4 HLA loci (preferably HLA-A, HLA-B, HLA-C, and HLA-DR) are typed. In some embodiments of ascertaining an HLA assignment, 4 HLA loci (preferably HLA-A, HLA-B, HLA-C, and HLA-DR) are typed. In some embodiments of ascertaining an HLA assignment, 6 HLA loci are typed. In some embodiments of ascertaining an HLA assignment, 8 HLA loci are typed.

In certain embodiments, preferably in addition to being restricted by an HLA allele shared with cells of the GBM, the population of allogeneic T cells comprising CMV-specific T cells shares at least 2 HLA alleles with cells of the GBM. In specific embodiments, the population of allogeneic T cells comprising CMV-specific T cells shares at least 2 out of 8 HLA alleles (for example, two HLA-A alleles, two HLA-B alleles, two HLA-C alleles, and two HLA-DR alleles) with cells of the GBM. In some embodiments, this sharing is ensured by ascertaining the HLA assignment of cells of the GBM, and selecting a population of allogeneic T cells comprising CMV-specific T cells (or a T cell line from which to derive the population of allogeneic T cells) that shares at least 2 (e.g., at least 2 out of 8) HLA alleles with such cells. In other embodiments, when ascertaining the HLA assignment of cells of the GBM is not possible (or is possible but not performed), this sharing is ensured by ascertaining the HLA assignment of the human patient (e.g., by using non-tumorous cells or tissue from the human patient), and selecting a population of allogeneic T cells comprising CMV-specific T cells (or a T cell line from which to derive the population of allogeneic T cells) that shares at least 2 (e.g., at least 2 out of 8) HLA alleles with the human patient.

The HLA assignment (i.e., the HLA loci type) can be ascertained (i.e., typed) by any method known in the art. Non-limiting exemplary methods for ascertaining the HLA assignment can be found in ASHI Laboratory Manual, Edition 4.2 (2003), American Society for Histocompatibility and Immunogenetics; ASHI Laboratory Manual, Supplements 1 (2006) and 2 (2007), American Society for Histocompatibility and Immunogenetics; Hurley, “DNA-based typing of HLA for transplantation.” in Leffell et al., eds., 1997, Handbook of Human Immunology, Boca Raton: CRC Press; Dunn, 2011, Int J Immunogenet 38:463-473; Erlich, 2012, Tissue Antigens, 80:1-11; Bontadini, 2012, Methods, 56:471-476; and Lange et al., 2014, BMC Genomics 15: 63.

In general, high-resolution typing is preferable for HLA typing. The high-resolution typing can be performed by any method known in the art, for example, as described in ASHI Laboratory Manual, Edition 4.2 (2003), American Society for Histocompatibility and Immunogenetics; ASHI Laboratory Manual, Supplements 1 (2006) and 2 (2007), American Society for Histocompatibility and Immunogenetics; Flomenberg et al., Blood, 104:1923-1930; Kögler et al., 2005, Bone Marrow Transplant, 36:1033-1041; Lee et al., 2007, Blood 110:4576-4583; Erlich, 2012, Tissue Antigens, 80:1-11; Lank et al., 2012, BMC Genomics 13:378; or Gabriel et al., 2014, Tissue Antigens, 83:65-75. In specific embodiments, the methods of treating GBM described herein further comprise prior to the administering step a step of ascertaining at least one HLA allele of cells of the GBM by high-resolution typing. In specific embodiments, the methods of treating GBM described herein further comprise prior to the administering step a step of ascertaining at least one HLA allele of the human patient by high-resolution typing.

The HLA allele by which the population of allogeneic T cells is restricted can be determined by any method known in the art, for example, as described in Trivedi et al., 2005, Blood 105:2793-2801; Barker et al., 2010, Blood 116:5045-5049; Hasan et al., 2009, J Immunol, 183:2837-2850; or Doubrovina et al., 2012, Blood 120:1633-1646.

Preferably, the HLA allele by which the population of allogeneic T cells is restricted and is shared with cells of the GBM is defined by high-resolution typing. Preferably, the HLA alleles that are shared between the population of allogeneic T cells and cells of the GBM are defined by high-resolution typing. Most preferably, both the HLA allele by which the population of allogeneic T cells is restricted and is shared with cells of the GBM, and the HLA alleles that are shared between the population of allogeneic T cells and cells of the GBM are defined by high-resolution typing.

### Obtaining or Generating a Population of Allogeneic T Cells Comprising CMV-Specific T Cells

The population of allogeneic T cells comprising CMV-specific T cells that is administered to the human patient can be generated by a method known in the art, or can be selected from a preexisting bank (collection) of cryopreserved T cell lines (each T cell line comprising CMV-specific T cells) generated by a method known in the art, and thawed and preferably expanded prior to administration. Preferably, unique identifier for each T cell line in the bank is associated with information as to which HLA allele(s) the respective T cell line is restricted, the HLA assignment of the respective T cell line, and/or the anti-CMV cytotoxic activity of the respective T cell line measured by a method known in the art (for example, as described in Trivedi et al., 2005, Blood 105:2793-2801; or Hasan et al., 2009, J Immunol 183: 2837-2850). The population of allogeneic T cells and the T cell lines in the bank are preferably obtained or generated by methods described below.

In various embodiments, the methods of treating GBM further comprise prior to the administering step a step of obtaining the population of allogeneic T cells.

In specific embodiments, the step of obtaining the population of allogeneic T cells comprises fluorescence activated cell sorting for CMV-positive T cells from a population of blood cells. In a specific embodiment, the population of blood cells are peripheral blood mononuclear cells (PBMCs) isolated from a blood sample(s) obtained from a human donor. The fluorescence activated cell sorting can be performed by any method known in the art, which normally involves staining the population of blood cells with an antibody that recognizes at least one CMV antigen before the sorting step.

In specific embodiments, the step of obtaining the population of allogeneic T cells comprises generating the population of allogeneic T cells in vitro. The population of allogeneic T cells can be generated in vitro by any method known in the art. Non-limiting exemplary methods of generating the population of allogeneic T cells can be found in Trivedi et al., 2005, Blood 105:2793-2801; Hasan et al., 2009, J Immunol 183: 2837-2850; Koehne et al., 2015, Biol Blood Marrow Transplant S1083-8791(15)00372-9, published online May 29, 2015; O'Reilly et al., 2007, Immunol Res 38:237-250; and O'Reilly et al., 2011, Best Practice & Research Clinical Haematology 24:381-391.

In certain embodiments, the step of generating the population of allogeneic T cells in vitro comprises sensitizing (i.e., stimulating) allogeneic T cells to one or more CMV antigens so as to produce CMV-specific T cells. In specific embodiments, the step of generating the population of allogeneic T cells in vitro comprises sensitizing allogeneic T cells to one or more CMV antigens presented by antigen presenting cells. The allogeneic T cells that are used for generating the population of allogeneic T cells in vitro can be isolated from the donor of the allogeneic T cells by any method known in the art, for example, as described in Trivedi et al., 2005, Blood 105:2793-2801; Hasan et al., 2009, J Immunol 183: 2837-2850; or O'Reilly et al., 2007, Immunol Res. 38:237-250. In a specific embodiment, the allogeneic T cells are enriched from peripheral blood lymphocytes separated from PBMCs of the donor of the allogeneic T cells. In a further specific embodiment, T cells are enriched from peripheral blood lymphocytes separated from PBMCs of the donor of the allogeneic T cells by depletion of adherent monocytes followed by depletion of natural killer cells. In various embodiments, the allogeneic T cells are cryopreserved for storage. In a specific embodiment, wherein the allogeneic T cells are cryopreserved, the cryopreserved allogeneic T cells are thawed and expanded in vitro before sensitizing. In a specific embodiment, wherein the allogeneic T cells are cryopreserved, the cryopreserved allogeneic T cells are thawed and then sensitized, but not expanded in vitro before sensitizing, and then optionally expanded. In specific embodiments, the allogeneic T cells are cryopreserved after sensitizing (sensitizing produces the CMV-specific T cells). In a specific embodiment, wherein the allogeneic T cells are cryopreserved after sensitizing, the cryopreserved allogeneic T cells are thawed and expanded in vitro to produce the population of allogeneic T cells comprising CMV-specific T cells. In another specific embodiment, wherein the allogeneic T cells are cryopreserved after sensitizing, the cryopreserved allogeneic T cells are thawed but not expanded in vitro to produce the population of allogeneic T cells comprising CMV-specific T cells. In other various embodiments, the allogeneic T cells are not cryopreserved. In a specific embodiment, wherein the allogeneic T cells are not cryopreserved, the allogeneic T cells are expanded in vitro before sensitizing. In a specific embodiment, wherein the allogeneic T cells are not cryopreserved, the allogeneic T cells are not expanded in vitro before sensitizing. In specific embodiments, the step of generating the population of allogeneic T cells in vitro further comprises, after sensitizing, cryopreserving the allogeneic T cells.

In specific embodiments, the methods of treating GBM described herein further comprise, before the administering step, steps of thawing cryopreserved CMV-antigen sensitized allogeneic T cells, and expanding the allogeneic T cells in vitro, to produce the population of allogeneic T cells.

In certain embodiments, the step of generating the population of allogeneic T cells in vitro comprises sensitizing allogeneic T cells using dendritic cells (preferably, the dendritic cells are derived from the donor of allogeneic T cells). In specific embodiments, the step of sensitizing allogeneic T cells using dendritic cells comprises loading the dendritic cells with at least one immunogenic peptide derived from one or more CMV antigens. In specific embodiments, the step of sensitizing allogeneic T cells using dendritic cells comprises loading the dendritic cells with a pool of overlapping peptides derived from one or more CMV antigens.

In certain embodiments, the step of generating the population of allogeneic T cells in vitro comprises sensitizing allogeneic T cells using cytokine-activated monocytes (preferably, the cytokine-activated monocytes are derived from the donor of allogeneic T cells). In specific embodiments, the step of sensitizing allogeneic T cells using cytokine-activated monocytes comprises loading the cytokine-activated monocytes with at least one immunogenic peptide derived from one or more CMV antigens. In specific embodiments, the step of sensitizing allogeneic T cells using cytokine-activated monocytes comprises loading the cytokine-activated monocytes with a pool of overlapping peptides derived from one or more CMV antigens.

In certain embodiments, the step of generating the population of allogeneic T cells in vitro comprises sensitizing allogeneic T cells using peripheral blood mononuclear cells (preferably, the peripheral blood mononuclear cells are derived from the donor of allogeneic T cells). In specific embodiments, the step of sensitizing allogeneic T cells using peripheral blood mononuclear cells comprises loading the peripheral blood mononuclear cells with at least one immunogenic peptide derived from one or more CMV antigens. In specific embodiments, the step of sensitizing allogeneic T cells using peripheral blood mononuclear cells comprises loading the peripheral blood mononuclear cells with a pool of overlapping peptides derived from one or more CMV antigens.

In certain embodiments, the step of generating the population of allogeneic T cells in vitro comprises sensitizing allogeneic T cells using an EBV-transformed B lymphocyte cell line (EBV-BLCL), for example, an EBV strain B95.8-transformed B lymphocyte cell line (preferably, the EBV-BLCL is derived from the donor of allogeneic T cells). The EBV-BLCL can be generated by any method known in the art, or as previously described in Trivedi et al., 2005, Blood 105:2793-2801 or Hasan et al., 2009, J Immunol 183:2837-2850. In specific embodiments, the step of sensitizing allogeneic T cells using an EBV-BLCL comprises loading the EBV-BLCL cells with at least one immunogenic peptide derived from one or more CMV antigens. In specific embodiments, the step of sensitizing allogeneic T cells using an EBV-BLCL comprises loading the EBV-BLCL cells with a pool of overlapping peptides derived from one or more CMV antigens.

In certain embodiments, the step of generating the population of allogeneic T cells in vitro comprises sensitizing allogeneic T cells using artificial antigen-presenting cells (AAPCs). In specific embodiments, the step of sensitizing allogeneic T cells using AAPCs comprises loading the AAPCs with at least one immunogenic peptide derived from one or more CMV antigens. In specific embodiments, the step of sensitizing allogeneic T cells using AAPCs comprises loading the AAPCs with a pool of overlapping peptides derived from one or more CMV antigens. In specific embodiments, the step of sensitizing allogeneic T cells using AAPCs comprises engineering the AAPCs to express at least one immunogenic CMV peptide or protein in the AAPCs.

In various embodiments, the pool of peptides is a pool of overlapping peptides spanning an antigen of CMV. In various embodiments, the pool of peptides is a pool of overlapping peptides spanning more than one antigen of CMV. In a specific embodiment, the pool of overlapping peptides is a pool of overlapping pentadecapeptides.

In specific embodiments, the population of allogeneic T cells has been cryopreserved for storage before administering. In specific embodiments, the population of allogeneic T cells has not been cryopreserved for storage before administering. In certain embodiments, the methods of treating GBM described herein further comprise, before the administering step, a step of thawing a cryopreserved form of the population of allogeneic T cells.

In various embodiments, the population of allogeneic T cells is derived from a T cell line. In specific embodiments, the T cell line has been cryopreserved for storage before administering. In specific embodiments, the T cell line has not been cryopreserved for storage before administering. In some embodiments, the T cell line has been expanded in vitro to derive the population of allogeneic T cells. In other embodiments, the T cell line has not been expanded in vitro to derive the population of allogeneic T cells. The T cell line can be sensitized to one or more CMV antigens (so as to produce CMV-specific T cells, for example, by a sensitizing step described above) before or after cryopreservation (if the T cell line has been cryopreserved), and before or after expanding in vitro (if the T cell line has been expanded in vitro). In certain embodiments, the methods of treating GBM described herein further comprise, before the administering step, a step of selecting the T cell line from a bank of a plurality of cryopreserved T cell lines (preferably each comprising CMV-specific T cells). Preferably, unique identifier for each T cell line in the bank is associated with information as to which HLA allele(s) the respective T cell line is restricted, and optionally also information as to the HLA assignment of the respective T cell line. In certain embodiments, the methods of treating GBM described herein further comprise, before the administering step, a step of thawing a cryopreserved form of the T cell line. In specific embodiments, the methods of treating GBM described herein further comprises, before the administering step, a step of expanding the T cell line (for example, after thawing a cryopreserved form of the T cell line) in vitro. The T cell line and the plurality of cryopreserved T cell lines can be generated by any method known in the art, for example, as described in Trivedi et al., 2005, Blood 105:2793-2801; Hasan et al., 2009, J Immunol 183: 2837-2850; Koehne et al., 2015, Biol Blood Marrow Transplant S1083-8791(15)00372-9, published online May 29, 2015; O'Reilly et al., 2007, Immunol Res 38:237-250; or O' Reilly et al., 2011, Best Practice & Research Clinical Haematology 24:381-391, or as describe above for generating the population of allogeneic T cells in vitro.

The population of allogeneic T cells comprising CMV-specific T cells that is administered to the human patient comprises CD8+ T cells, and in a specific embodiment also comprises CD4+ T cells.

The CMV-specific T cells administered in accordance with the methods described herein recognize at least one antigen of CMV. In specific embodiments, the CMV-specific T cells administered in accordance with the methods described herein recognize a CMV antigen expressed on cells of the GBM (e.g., a CMV antigen known to be expressed on cells of the GBM by those skilled in the art). In a further specific embodiment, the CMV-specific T cells administered in accordance with the methods described herein recognize a CMV antigen uniquely expressed on cells of the GBM and not expressed on non-tumorous cells of the human patient. In another further specific embodiment, the CMV-specific T cells administered in accordance with the methods described herein recognize a CMV antigen expressed at a higher level on cells of the GBM relative to non-tumorous cells of the human patient. In specific embodiments, the CMV-specific T cells administered in accordance with the methods described herein recognize CMVpp65. In specific embodiments, the CMV-specific T cells administered in accordance with the methods described herein recognize CMV IE1.

### Administration and Dosage

The route of administration of the population of allogeneic T cells and the amount to be administered to the human patient can be determined based on the condition of the human patient and the knowledge of the physician. Generally, the administration is intravenous.

In certain embodiments, the administering is by infusion of the population of allogeneic T cells. In some embodiments, the infusion is bolus intravenous infusion. In certain embodiments, the administering comprises administering at least about 1×10⁵ T cells of the population of allogeneic T cells per kg per dose per week to the human patient. In some embodiments, the administering comprises administering about 1×10⁵ to about 5×10⁵ T cells of the population of allogeneic T cells per kg per dose per week to the human patient. In some embodiments, the administering comprises administering about 5×10⁵ to about 1×10⁶ T cells of the population of allogeneic T cells per kg per dose per week to the human patient. In some embodiments, the administering comprises administering about 1×10⁶ to about 5×10⁶ T cells of the population of allogeneic T cells per kg per dose per week to the human patient. In some embodiments, the administering comprises administering about 2×10⁶ to about 5×10⁶ T cells of the population of allogeneic T cells per kg per dose per week to the human patient. In some embodiments, the administering comprises administering about 5×10⁶ to about 1×10⁷ T cells of the population of allogeneic T cells per kg per dose per week to the human patient. In some embodiments, the administering comprises administering about 1×10⁶ to about 1×10⁷ T cells of the population of allogeneic T cells per kg per dose per week to the human patient. In some embodiments, the administering comprises administering about 1×10⁶ to about 5×10⁶ T cells of the population of allogeneic T cells per kg per dose per week to the human patient. In some embodiments, the administering comprises administering about 1×10⁶ to about 2×10⁶ T cells of the population of allogeneic T cells per kg per dose per week to the human patient. In a specific embodiment, the administering comprises administering about 1×10⁶ T cells of the population of allogeneic T cells per kg per dose per week to the human patient. In another specific embodiment, the administering comprises administering about 2×10⁶ T cells of the population of allogeneic T cells per kg per dose per week to the human patient.

In certain embodiments, the methods of treating GBM described herein comprise administering at least 2 doses of the population of allogeneic T cells to the human patient. In specific embodiments, the methods of treating GBM described herein comprise administering 2, 3, 4, 5, or 6 doses of the population of allogeneic T cells to the human patient.

In certain embodiments, the methods of treating GBM described herein comprise administering a first cycle of one dose per week of the population of allogeneic T cells for 3 consecutive weeks followed by a washout period during which no dose of the population of allogeneic T cells is administered, followed by a second cycle of the one dose per week of the population of allogeneic T cells for 3 consecutive weeks. In certain embodiments, the methods of treating GBM described herein comprise administering at least two cycles of one dose per week of the population of allogeneic T cells for 3 consecutive weeks, each cycle separated by a washout period during which no dose of the population of allogeneic T cells is administered. In specific embodiments, the methods of treating GBM described herein comprise administering two, three, four, five, or six cycles of one dose per week of the population of allogeneic T cells for 3 consecutive weeks, each cycle separated by a washout period during which no dose of the population of allogeneic T cells is administered. In a specific embodiment, the washout period is about three weeks. Preferably, an additional cycle is administered only when the previous cycle has not exhibited toxicity (for example, no grade 3-5 serious adverse events, graded according to NCI CTCAE 4.0).

In certain embodiments, a first dosage regimen described herein is carried out for a first period of time, followed by a second and different dosage regimen described herein that is carried out for a second period of time, wherein the first period of time and the second period of time are optionally separated by a washout period (for example, about three weeks). Preferably, the second dosage regimen is carried out only when the first dosage regimen has not exhibited toxicity (for example, no grade 3-5 serious adverse events, graded according to NCI CTCAE 4.0).

The term “about” shall be construed so as to allow normal variation.

### Patients

The human patient can be anyone who has glioblastoma multiforme (GBM) or is suspected of having GBM. In preferred embodiments, the GBM of the human patient to be treated expresses one or more CMV antigens. In certain embodiments, the GBM of the human patient to be treated is analyzed for the presence of one or more CMV antigens before the administering step. In certain embodiments, the human patient has been diagnosed with GBM by any method known in the art (e.g., by magnetic resonance imaging, computerized tomography scan, stereostactic biopsy, craniotomy with tumor resection, and/or pathological examination). In certain embodiments, the human patient has not been diagnosed with GBM, but is suspected of having GBM. In certain embodiments, the human patient has one or more symptoms associated with the GBM, including, for example and without limitation, seizure, nausea, vomiting, headache, memory loss, hemiparesis, progressive memory deficit, personality deficit, and/or neurological deficit. In specific embodiments, the human patient is asymptomatic.

### Incorporation by Reference

Various publications are cited herein, the disclosures of which are hereby incorporated by reference herein in their entireties.

What is claimed is:

1. A method of treating glioblastoma multiforme (GBM) in a human patient in need thereof, comprising administering to the human patient a population of allogeneic T cells comprising CMV (cytomegalovirus)-specific T cells.

2. The method of claim 1, wherein the population of allogeneic T cells is restricted by an HLA allele shared with cells of the GBM.

3. The method of claim 1 or 2, further comprising prior to said administering step a step of ascertaining at least one HLA allele of cells of the GBM by high-resolution typing.

4. The method of any of claims 1-3, wherein the population of allogeneic T cells shares at least 2 out of 8 HLA alleles with cells of the GBM.

5. The method of claim 4, wherein the 8 HLA alleles are two HLA-A alleles, two HLA-B alleles, two HLA-C alleles, and two HLA-DR alleles.

6. The method of any of claims 1-5, wherein the CMV-specific T cells recognize CMVpp65.

7. The method of any of claims 1-5, wherein the CMV-specific T cells recognize CMV IE1.

8. The method of any of claims 1-7, which further comprises prior to said administering step a step of generating the population of allogeneic T cells in vitro.

9. The method of claim 8, wherein the step of generating the population of allogeneic T cells in vitro comprises sensitizing allogeneic T cells to one or more CMV antigens.

10. The method of claim 8, wherein the step of generating the population of allogeneic T cells in vitro comprises sensitizing allogeneic T cells using dendritic cells, cytokine-activated monocytes, peripheral blood mononuclear cells, or EBV-BLCL (EBV-transformed B lymphocyte cell line) cells.

11. The method of claim 10, wherein the step of sensitizing allogeneic T cells using dendritic cells, cytokine-activated monocytes, or peripheral blood mononuclear cells comprises loading the dendritic cells, the cytokine-activated monocytes, the peripheral blood mononuclear cells, or the EBV-BLCL cells with at least one immunogenic peptide derived from one or more CMV antigens.

12. The method of claim 10, wherein the step of sensitizing allogeneic T cells using dendritic cells, cytokine-activated monocytes, or peripheral blood mononuclear cells comprises loading the dendritic cells, the cytokine-activated monocytes, the peripheral blood mononuclear cells, or the EBV-BLCL cells with a pool of overlapping peptides derived from one or more CMV antigens.

13. The method of claim 8, wherein the step of generating the population of allogeneic T cells in vitro comprises sensitizing allogeneic T cells using artificial antigen presenting cells (AAPCs).

14. The method of claim 13, wherein the step of sensitizing allogeneic T cells using AAPCs comprises loading the AAPCs with at least one immunogenic peptide derived from one or more CMV antigens.

15. The method of claim 13, wherein the step of sensitizing allogeneic T cells using AAPCs comprises loading the AAPCs with a pool of overlapping peptides derived from one or more CMV antigens.

16. The method of claim 13, wherein the step of sensitizing allogeneic T cells using AAPCs comprises engineering the AAPCs to express at least one immunogenic CMV peptide or protein in the AAPCs.

17. The methods of claim 12 or 15, wherein the pool of overlapping peptides is a pool of overlapping pentadecapeptides.

18. The method of any of claims 9-17, which further comprises, after sensitizing, cryopreserving the allogeneic T cells.

19. The method of any of claims 1-18, which further comprises, before the administering step, steps of thawing cryopreserved CMV-antigen sensitized allogeneic T cells, and expanding the allogeneic T cells in vitro, to produce the population of allogeneic T cells.

20. The method of any of claims 1-19, which further comprises, before the administering step, a step of thawing a cryopreserved form of the population of allogeneic T cells.

21. The method of any of claims 1-17, wherein the population of allogeneic T cells is derived from a T cell line.

22. The method of claim 21, which further comprises, before the administering step, a step of selecting the T cell line from a bank of a plurality of cryopreserved T cell lines.

23. The method of claim 21 or 22, which further comprises, before the administering step, a step of thawing a cryopreserved form of the T cell line.

24. The method of any of claims 21-23, which further comprises, before the administering step, a step of expanding the T cell line in vitro.

25. The method of any of claims 1-24, wherein the administering is by infusion of the population of allogeneic T cells.

26. The method of claim 24, wherein the infusion is bolus intravenous infusion.

27. The method of any of claims 1-26, wherein the administering comprises administering at least about 1×10⁵ T cells of the population of allogeneic T cells per kg per dose per week to the human patient.

28. The method of any of claims 1-26, wherein the administering comprises administering about 1×10⁶ to about 2×10⁶ T cells of the population of allogeneic T cells per kg per dose per week to the human patient.

29. The method of any of claims 1-26, wherein the administering comprises administering about 1×10⁶ T cells of the population of allogeneic T cells per kg per dose per week to the human patient.

30. The method of any of claims 1-26, wherein the administering comprises administering about 2×10⁶ T cells of the population of allogeneic T cells per kg per dose per week to the human patient.

31. The method of any of claims 1-30, wherein the administering comprises administering at least 2 doses of the population of allogeneic T cells to the human patient.

32. The method of claim 31, wherein the administering comprises administering 2, 3, 4, 5, or 6 doses of the population of allogeneic T cells to the human patient.

33. The method of any of claims 1-30, wherein the administering comprises administering a first cycle of one dose per week of the population of allogeneic T cells for 3 consecutive weeks followed by a washout period during which no dose of the population of allogeneic T cells is administered, followed by a second cycle of said one dose per week of the population of allogeneic T cells for 3 consecutive weeks.

34. The method of any of claims 1-30, wherein the administering comprises administering two, three, four, five, or six cycles of one dose per week of the population of allogeneic T cells for 3 consecutive weeks, each cycle separated by a washout period during which no dose of the population of allogeneic T cells is administered.

35. The method of claim 33 or 34, wherein the washout period is about three weeks.
